(19) United States
(12) Patent Application Publication
(10) Pub. No.: US 2023/0157970 A1
(43) Pub. Date: May 25, 2023

(54) Targeted Therapy of Kidney Fibrosis

(71) Applicant: Board of Regents, The University of Texas System, Austin, TX (US)

(72) Inventors: Md Nurunnabi, El Paso, TX (US); Humayra Afrin, El Paso, TX (US)

(21) Appl. No.: 18/057,567

(22) Filed: Nov. 21, 2022

Related U.S. Application Data
(60) Provisional application No. 63/264,295, filed on Nov. 19, 2021.

(51) Int. Cl. A61K 9/51; A61K 9/00; A61K 31/5377; A61K 31/635; A61K 31/437

(52) U.S. Cl. CPC A61K 9/5153; A61K 9/0019; A61K 31/5377; A61K 31/635; A61K 31/437; A61K 9/5146; B82Y 5/00

(57) ABSTRACT

Procedures for targeted treatment of kidney fibrosis include administering to a subject in need of treatment of kidney fibrosis a compound including a plurality of nanoparticle including at least one BCL-2/BCL-xl inhibitor; at least one polymer comprising a member selected from the group consisting of PLGA, PEG or PVA; and at least one targeting peptide, wherein each of the plurality of nanoparticles defines a hollow shell formed by the polymer, the at least one BCL-2/BCL-xl inhibitor is located within an interior of the hollow shell, and the at least one targeting peptide is coupled to an exterior surface of the hollow shell.

### CROSS REFERENCE TO RELATED APPLICATION

This application is a utility conversion and claims priority to U.S. Ser. No. 63/264,295, filed Nov. 19, 2021, the entire contents of which are hereby incorporated herein by reference for all purposes.

### BACKGROUND INFORMATION

### 1. Field

The present invention relates generally to the field of medicine and disease treatment. More particularly, it concerns methods and compositions for targeted therapy of kidney fibrosis.

### 2. Background

It is known to those of skill in the art that bcl2/BCL-xl inhibitors can improve fibrosis such as skin fibrosis and kidney fibrosis. Both oral and intravenous formulation of bcl2/BCL-xl inhibitors can produce therapeutic effect in fibrosis.

An example of a BCL-2 inhibitor is angiotensin. FIG. 1 illustrates the renin angiotensin system. The liver 110, kidney 120, lungs 130 and heart 140 are all involved in the body's inhibition of fibrosis via renin angiotensin.

However, a very common problem associated with intravenous administration of BCL-2 inhibitors is thrombocytopenia. Low platelet levels are symptomatic of thrombocytopenia and can occur when bone marrow does not make enough platelets. Meanwhile, oral administration is not as effective.

Heretofore, the requirements of ameliorating kidney fibrosis while avoiding side effects and other undesirable consequences have not been fully met. In view of the foregoing, there is a need in the art for a solution that simultaneously solves all of these problems.

### SUMMARY

An overall goal of embodiments of the present disclosure is to ameliorate kidney fibrosis with minimum toxicity and more therapeutic effect by using kidney targeting nanoparticle conjugated drugs. More specifically, a plurality of nanoparticles are intravenously injected that include at least one BCL-2/BCL-xl inhibitor.

An illustrative embodiment of the present disclosure is a composition of matter for targeted treatment of kidney fibrosis, comprising: a plurality of nanoparticle comprising at least one BCL-2/BCL-xl inhibitor; at least one polymer; and at least one targeting peptide.

Another illustrative embodiment of the present disclosure is a method of targeted treatment of kidney fibrosis, comprising: administering to a mammal in need thereof a plurality of nanoparticles comprising at least one BCL-2/BCL-xl inhibitor; at least one polymer; and at least one targeting peptide.

Another illustrative embodiment of the present disclosure is a composition of matter for targeted treatment of kidney fibrosis, comprising: a plurality of nanoparticle comprising at least one BCL-2/BCL-xl inhibitor; at least one polymer comprising a member selected from the group consisting of PLGA, PEG or PVA; and at least one targeting peptide, wherein each of the plurality of nanoparticles defines a hollow shell formed by the polymer, the at least one BCL-2/BCL-xl inhibitor is located within an interior of the hollow shell, and the at least one targeting peptide is coupled to an exterior surface of the hollow shell.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 illustrates a schematic diagram of the renin angiotensin system appropriately labeled prior art.

FIG. 2 illustrates components of a nanoparticle in accordance with the invention.

FIGS. 3A-3B illustrate particle characterization in accordance with the invention.

FIGS. 4A-4H illustrate biodistribution of kidney targeting particles in accordance with the invention.

FIG. 5 illustrates kidneys after harvesting from different treatment groups in accordance with the invention.

FIGS. 6A-6B illustrate average size and weight of kidneys after harvesting from different treatment groups in accordance with the invention.

FIGS. 7A-7F illustrate analysis of kidneys after harvesting from different treatment groups in accordance with the invention.

FIG. 8 illustrates expressions from kidneys after harvesting from different treatment groups in accordance with the invention.

FIGS. 9A-9R illustrate apoptosis from kidneys after harvesting from different treatment groups in accordance with the invention.

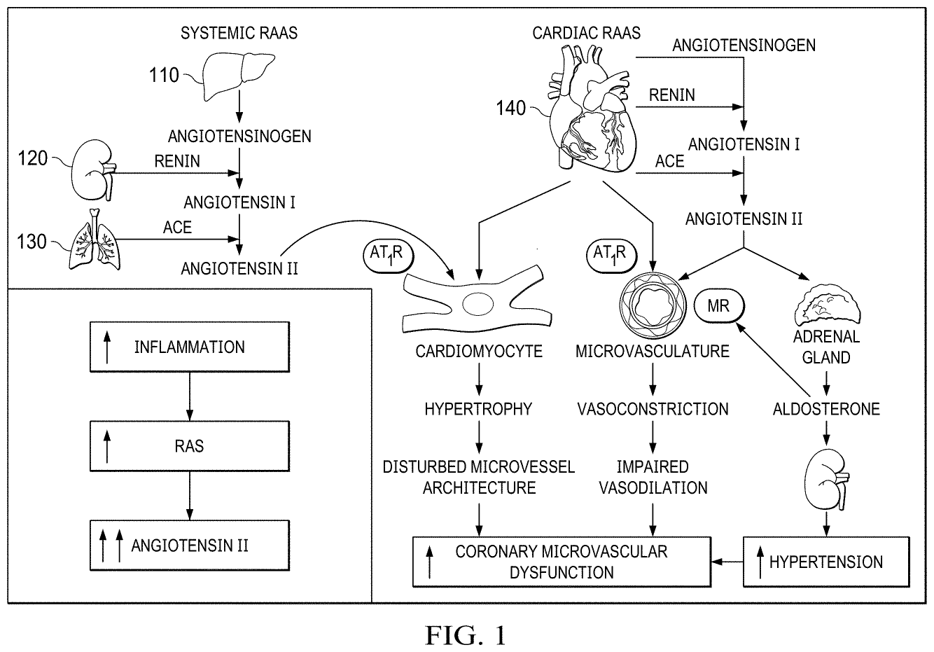

### DETAILED DESCRIPTION

The disclosure of this application is technically related to co-pending U.S. Ser. No. ______ (attorney docket number UTEP2022-005-1), filed Nov. 21, 2022, the entire contents of which are hereby expressly incorporated by reference for all purposes.

Embodiments of this disclosure include methods and formulations that will ameliorate kidney fibrosis. While not being bound by theory, the methods and formulations induce apoptosis of fibrosed cells.

Embodiments of this disclosure include nanoparticles having a polymer, a bcl2/BCL-xl inhibitor, and a kidney targeting peptide. The peptide is coupled to the polymer and targets the kidneys. The polymer is coupled to the inhibitor and reduces thrombocytopenia. The bcl2/BCL-xl inhibitor ameliorates kidney fibrosis.

The polymer can be formed as a hollow shell. The hollow shell can be a nanoparticle. The bcl2a/BCL-xl inhibitor can be located within the hollow shell. The peptide can be coupled to an exterior surface of the hollow shell. This configuration overcomes the thrombocytopenia which is a very common problem associated with IV administration of BCL-2 inhibitors. This is an important medical and commercial advantage of embodiments of this disclosure.

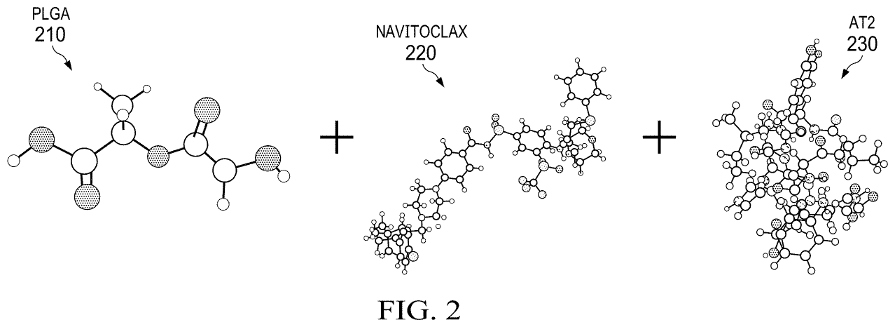

Referring to FIG. 2, an embodiment of this disclosure includes a nanoparticle including a PLGA component 210, a Navitoclax component 220, and an AT2 component 230. This is an exemplary nanoparticle in accordance with an embodiment of the present disclosure. This exemplary nanoparticle can be made by water in oil emulsion.

The components of the formulation compound can be substituted partially or wholly with other components that provide equivalent functionality. Examples of components that can be substituted include the following.

### List of BCL-2 Inhibitors:

| Inhibitors Name | CAS |
|---|---|
| Navitoclax | 923564-51-6 |
| ABT-737 | 852808-04-9 |
| Obatoclax Mesylate | 803712-79-0 |
| TW-37 | 877877-35-5 |
| Venetoclax | 1257044-40-8 |
| BM-1074 | 1391108-10-3 |
| Gambogic Acid | 2752-65-0 |

### List of Polymers:

PLGA: Poly(D,L-lactide-co-glycolide)
PEG: Poly(ethylene glycol)
PVA: Polyvinyl alcohol

### List of Targeting Peptides:

| Peptide name | Sequence |
|---|---|
| Angiotensin II targeting | RVYIHPI |
| (AT II aka AT2) | |
| VCam1 | VHPKQHRGGSKGC |
| EpCAM | DARPin C9 |

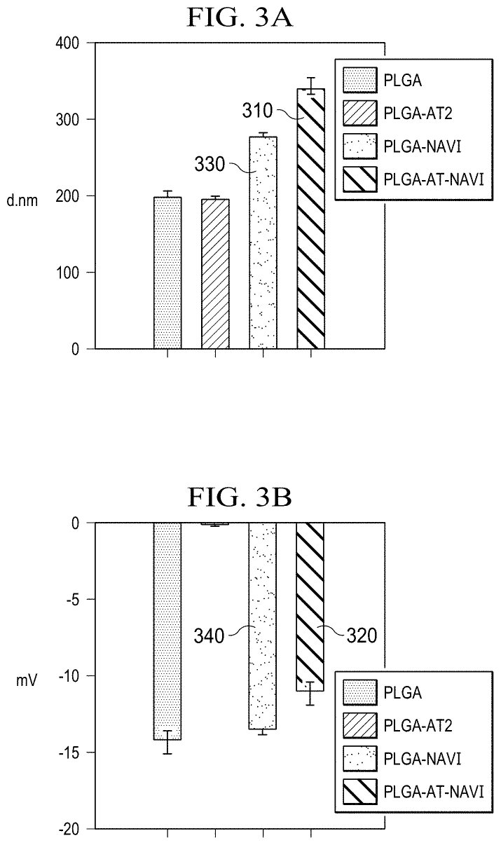

FIGS. 3A-3B illustrate size (FIG. 3A) and Zeta potential (FIG. 3B) of PLGA, PLGA-AT2, PLGA-NAVI, and PLGA-AT-NAVI nanoparticle particles made by water in oil emulsion were measured by Zetasizer (Malvern nanoseries, USA). The average size and zeta of PLGA-AT2-NAVI nanoparticles were 339.6667 nm 310 and −11.1333 mV 320. Whereas the average size and zeta potential of PLGA-AT2 nanoparticles were 196.5 nm 330 and −0.123 mV 340. This shows that targeted kidney fibrosis treatment with PLGA-AT2-NAVI nanoparticles is appropriate.

### EXAMPLES

Specific exemplary embodiments will now be further described by the following, nonlimiting examples which will serve to illustrate in some detail various features. The following examples are included to facilitate an understanding of ways in which embodiments of the present disclosure may be practiced. However, it should be appreciated that many changes can be made in the exemplary embodiments which are disclosed while still obtaining like or similar result without departing from the scope of embodiments of the present disclosure. Accordingly, the examples should not be construed as limiting the scope of the present disclosure.

### Example 1

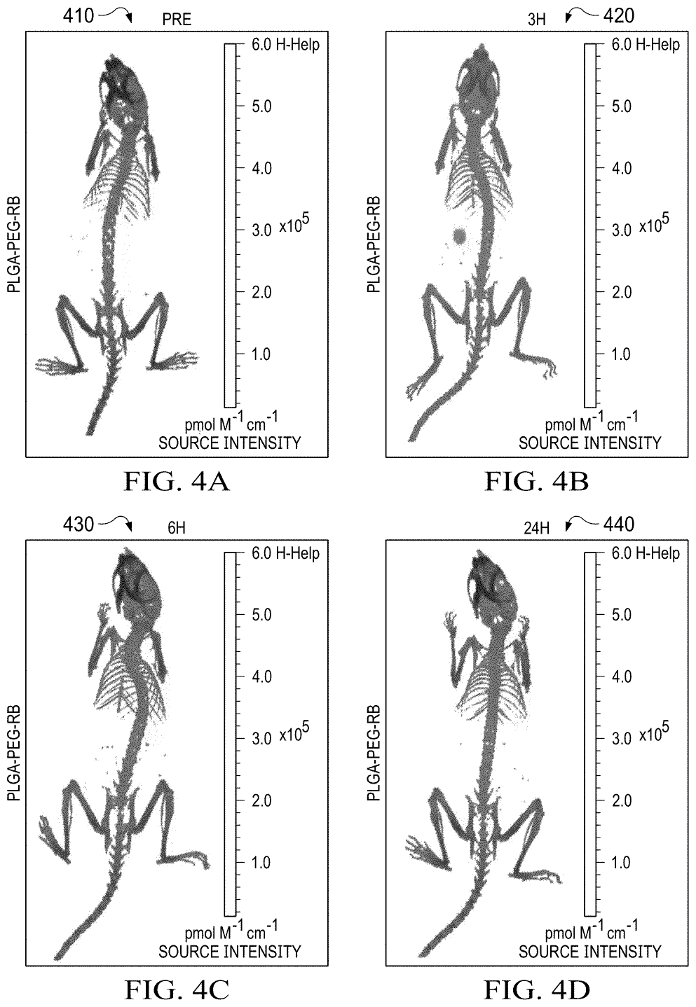

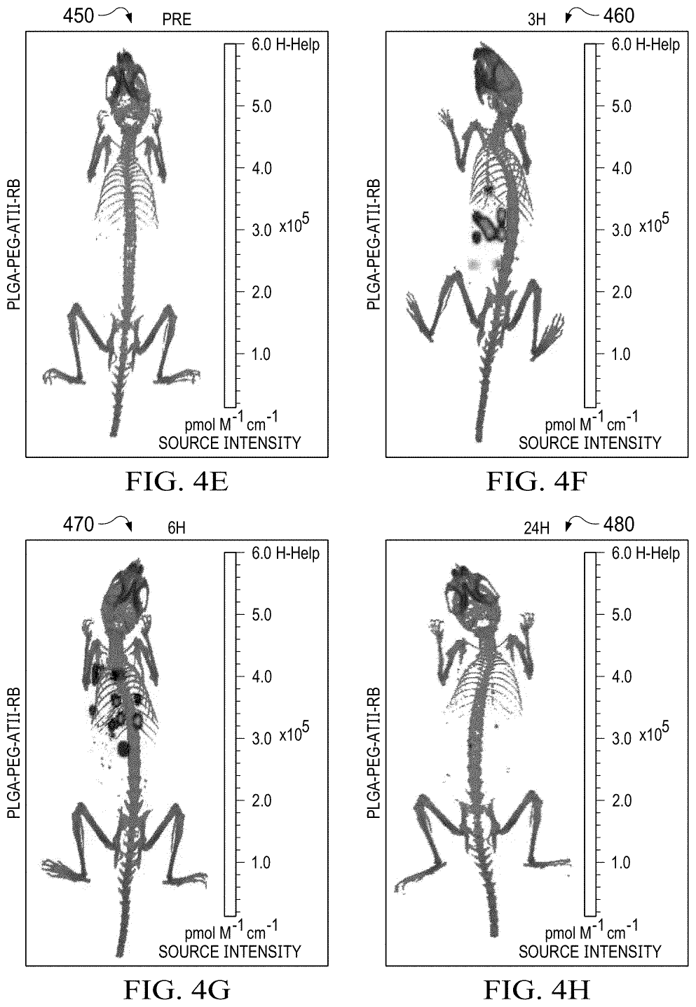

FIGS. 4A-4 H illustrate results of a biodistribution of kidney targeting particle experiment. A control group was tail vein injected with PLGA-PEG-RB. The concentration of sample was 1 mg/Kg. There was IVIS and CT co-registration.

The acronym RB represents an x-ray imaging marker. FIG. 4A shows a pre-injection image 410 for a specimen from the control group. FIG. 4B shows a 3 hour image 420 for the specimen from the control group. FIG. 4C shows a 6 hour image 410 for the specimen from the control group. FIG. 4D shows a 24 hour image 440 for the specimen from the control group.

An experimental group was tail vein injected with PLGA-PEG-ATII-RB. FIG. 4E shows a pre-injection image 450 for a specimen from the control group. FIG. 4F shows a 3 hour image 460 for the specimen from the group. FIG. 4G shows a 6 hour image 470 for the specimen from the group. FIG. 4H shows a 24 hour image 480 for the specimen from the group. This shows that the PLGA-PEG-ATII-RB nanoparticles accumulate in the kidney for at least 3-6 hours. Consequently, embodiments of this disclosure ameliorate kidney fibrosis with minimum toxicity and more therapeutic effect compared to the control group.

### Example 2

This example was an animal study that created a CKD model by injecting 250 mg/kg folic acid intraperitonially (single injection). After 15 days the treatment started. The mice were divided in 5 groups of untreated, healthy, Fausidil, PLGA-Navitoclax, and PLGA-AT2-Navitoclax.

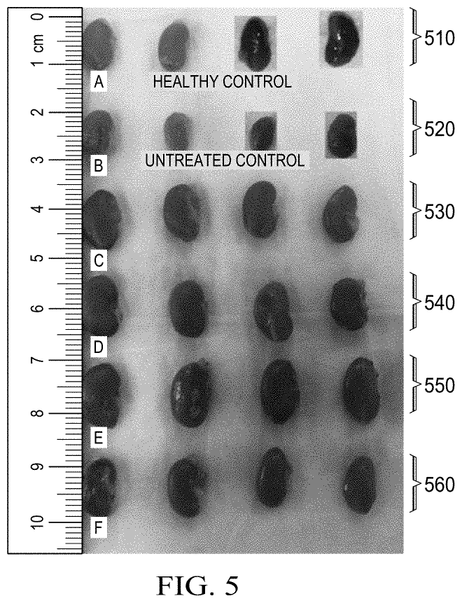

FIG. 5: Figure: Image of the kidney after harvesting. The kidney from different treatment groups: A-Healthy control 510, B-Untreated control 520, C-Navi(10 mg/kg oral) 530, D-Fausidil(10 mg/kg IP) 540, E-PLGA-Navi(10 mg/kg IV) 550, F-PLGA-AT2-Navi(10 mg/kg IV) 560.

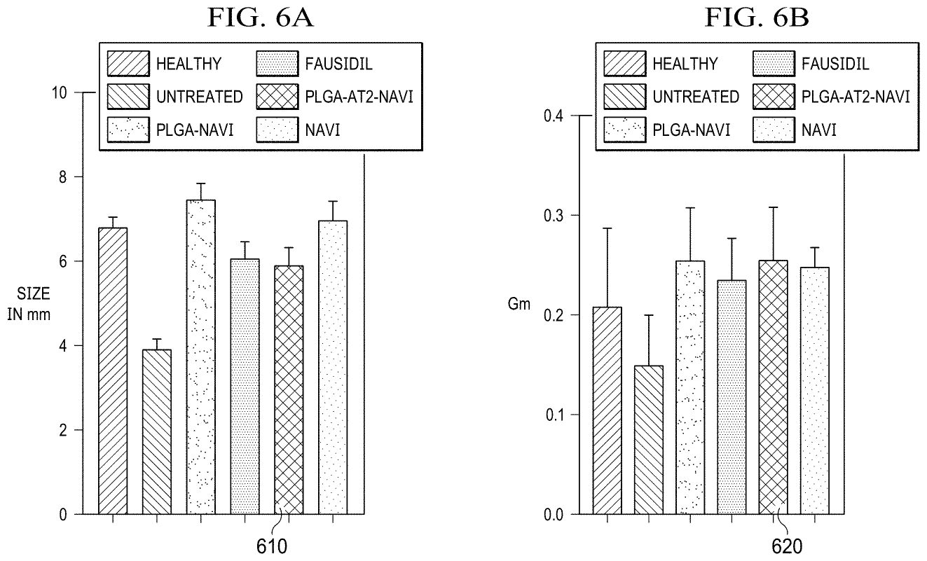

FIGS. 6A-6B: The size of the untreated kidney shrunk by 42.46% (p-value 0.0062) than the healthy control (A). After treatment with Navi(10 mg/kg oral), Fausidil (10 mg/kg IP), PLGA-Navi(10 mg/kg IV), and PLGA-AT2-Navi(10 mg/kg IV) 610 size of the kidney increased up to 47.52% and 43.95% with PLGA-Navitoclax and oral Navitoclax. With our kidney targeting particle PLGA-AT2-Navitoclax the size increased by 33.68%. The weight of kidney (B) was also measured in gm and the weight 620 of the kidney treated with PLGA-AT2-Navi.

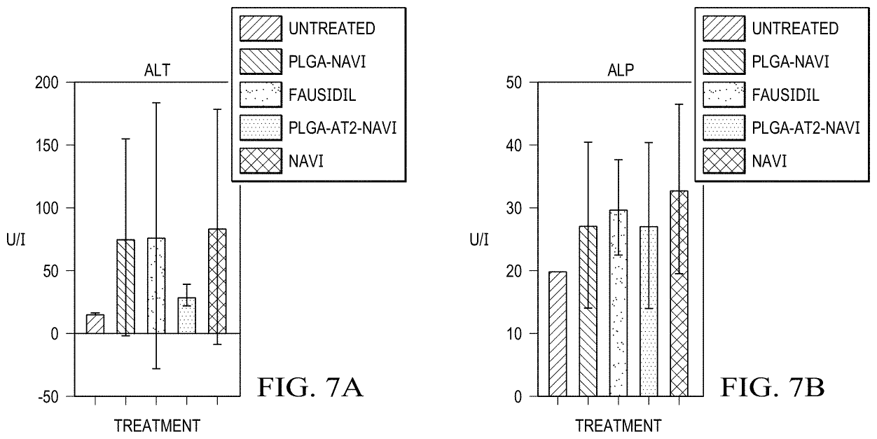

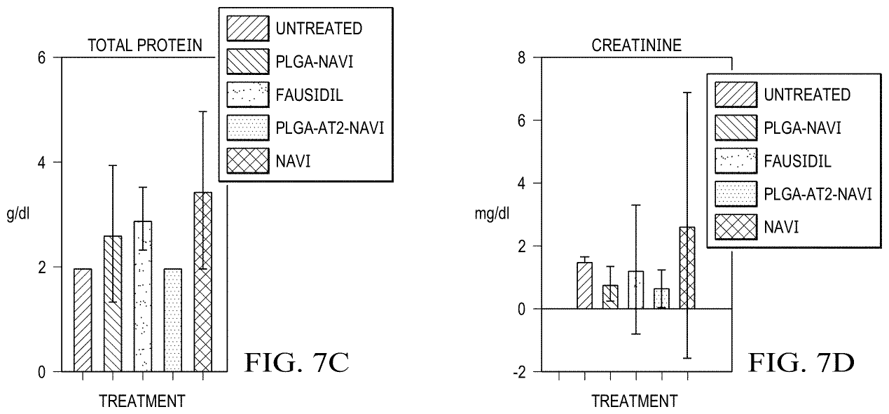

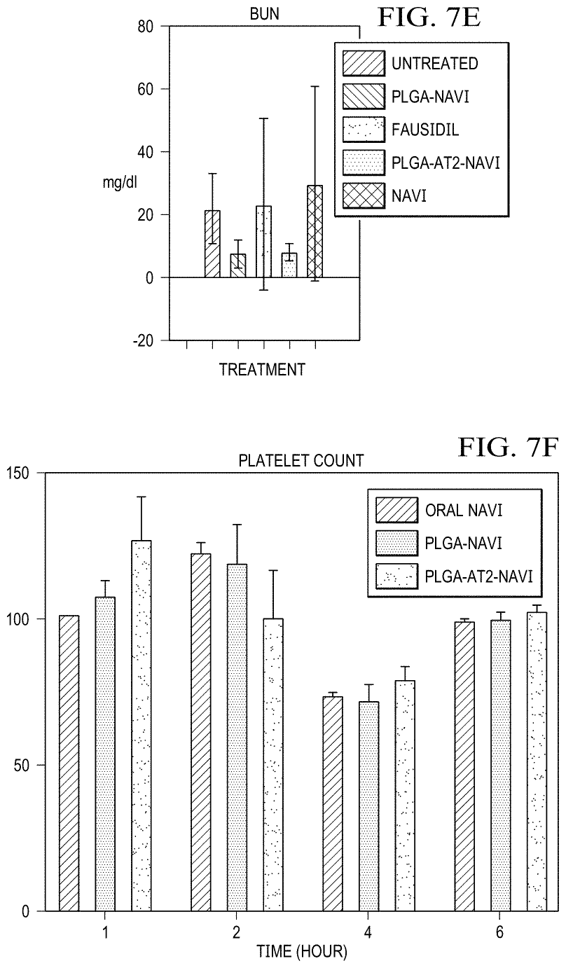

FIGS. 7A-7F: Serum analysis of the treated mice group (A-E). No liver or kidney toxicity was detected in the serum analysis collected from the mice. PLGA-AT2-Navi treatment group shows less liver (Alanine Aminotransferase (A), Alkaline Phosphatase (B), total protein (C)) and kidney (creatinine (D), blood urea nitrogen (E)) toxicity marker. In blood analysis for 6 hours of treatment shows (F) that there is no significant thrombocytopenia in PLGA-AT2-Navi group then oral Navi (p-value>0.999).

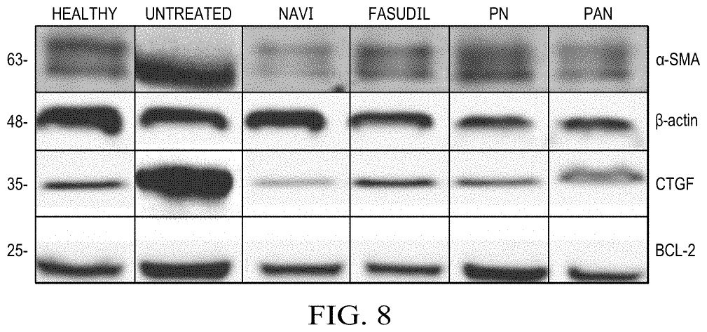

FIG. 8: Western blot analysis from the kidney sample. Bcl2 expression was decreased in the sample collected from different group Healthy control, Untreated control, Navi(10 mg/kg oral), Fausidil(10 mg/kg IP), PLGA-Navi(10 mg/kg IV), PLGA-AT2-Navi(10 mg/kg IV) of mice. Oral Navitoclax and PLGA-AT2-Navitoclax group had less expression of bcl2 then other groups. As a marker of fibrosis, we measured α-Smooth muscle actin (α-SMA) and Connective tissue growth factor (CTGF) protein expression and both factor has been decreased significantly in the treatment groups than control.

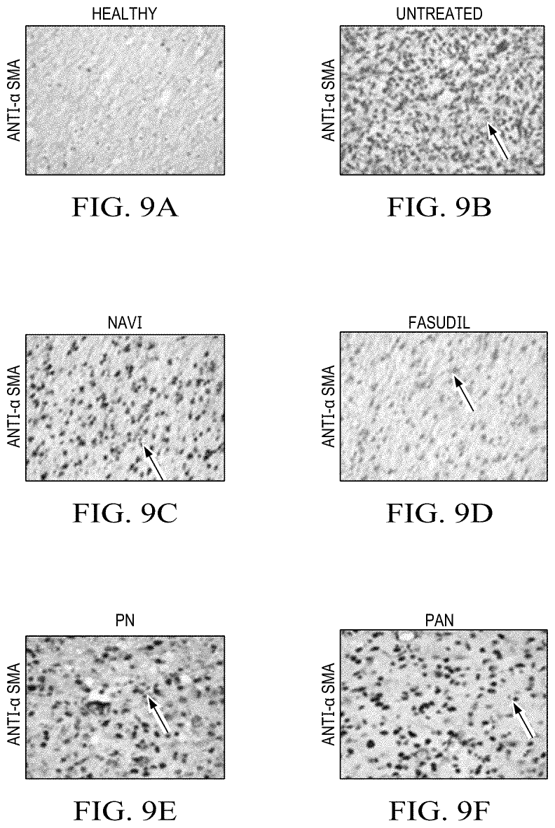

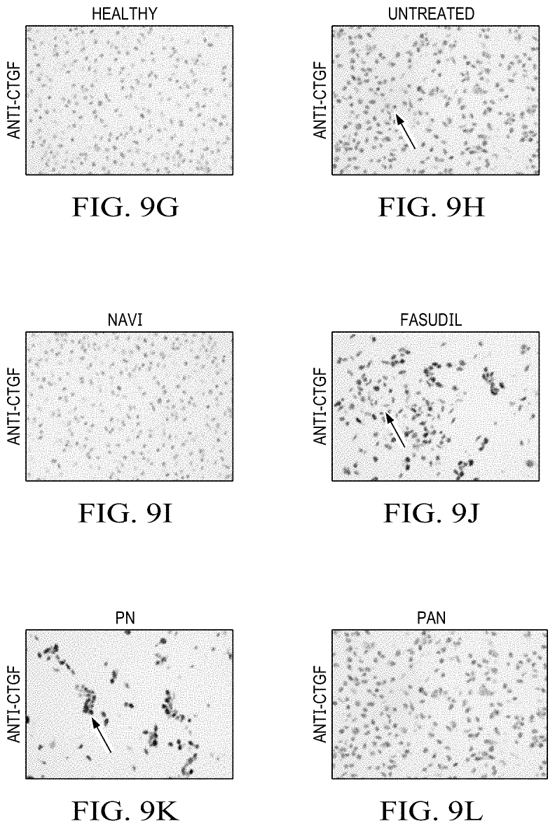

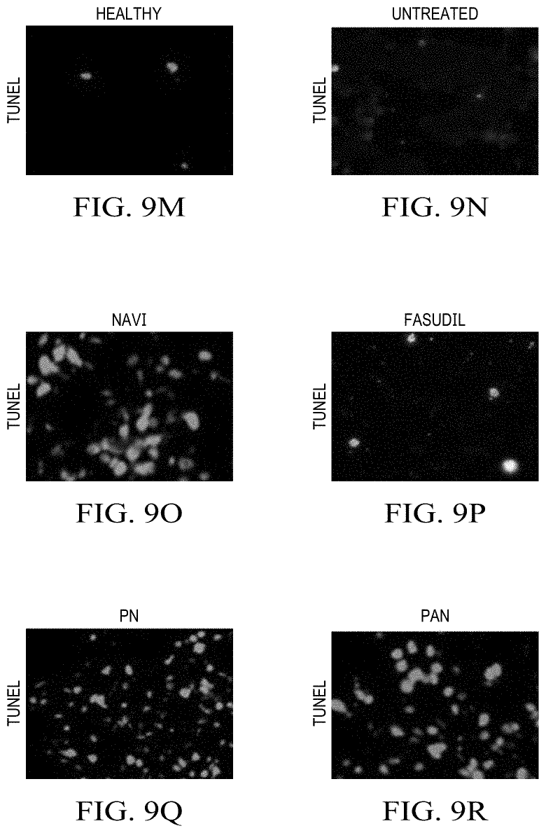

FIGS. 9A-9R: Immunohistochemistry (IHC) and TUNEL assay of kidney section. IHC for alpha-SMA protein of different treatment group (A-F). The arrow pointed to the dark dot which represented the alpha-SMA protein. It shows that in the PLGA-AT2-Navi treatment group the alpha-SMA was reduced significantly than the untreated group. IHC of CTGF (connective Tissue Growth Factor) also shows a reduced expression of CTGF in the treatment group with PLGA-AT2-Navi. The dark dot from FIGS. 9G-9L represented the CTGF protein. The arrow showed the CTGF protein. FIGS. 9M-9R showed the broken nucleus of DNA (apoptosis) done by TUNEL assay. With PLGA-AT2-Navi, PLGA-AT2, and Navi treatment groups apoptosis was observed.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A composition of matter for targeted treatment of kidney fibrosis, comprising:
a plurality of nanoparticles comprising
at least one BCL-2/BCL-xl inhibitor;
at least one polymer; and
at least one targeting peptide.

2. The composition of claim 1, wherein the at least one BCL-2/BCL-xl inhibitor comprises at least one BCL-2 inhibitor selected from the group consisting of Navitoclax, ABT-737, Venetoclax or BM-1074.

3. The composition of claim 1, wherein the at least one BCL-2/BCL-xl inhibitor comprises at least one BCL-2 inhibitor selected from the group consisting of Navitoclax, ABT-737, Obatoclax Mesylate, TW-37, Venetoclax, BM-1074 or Gambogic Acid.

4. The composition of claim 1, wherein the at least one polymer comprises a member selected from the group consisting of PLGA, PEG or PVA.

5. The composition of claim 1, wherein the at least one targeting peptide comprises AT2.

6. The composition of claim 1, wherein the at least one targeting peptide comprises at least one member selected from the group consisting of AT2, VCam1 or EpCAM.

7. The composition of claim 1, wherein each of the plurality of nanoparticles defines a hollow shell formed by the polymer, the at least one BCL-2/BCL-xl inhibitor is located within an interior of the hollow shell, and the at least one targeting peptide is coupled to an exterior surface of the hollow shell.

8. A method of targeted treatment of kidney fibrosis, comprising:
administering to a mammal in need thereof a plurality of nanoparticles comprising
at least one BCL-2/BCL-xl inhibitor;
at least one polymer; and
at least one targeting peptide.

9. The method of claim 8, wherein administering includes an intravenous injection.

10. The method of claim 8, wherein administering includes administering nanoparticles wherein the at least one BCL-2/BCL-xl inhibitor comprises at least one BCL-2 inhibitor selected from the group consisting of Navitoclax, ABT-737, Venetoclax or BM-1074.

11. The method of claim 8, wherein administering includes administering nanoparticles wherein the at least one BCL-2/BCL-xl inhibitor comprises at least one BCL-2 inhibitor selected from the group consisting of Navitoclax, ABT-737, Obatoclax Mesylate, TW-37, Venetoclax, BM-1074 or Gambogic Acid.

12. The method of claim 8, wherein administering includes administering nanoparticles wherein the at least one polymer comprises a member selected from the group consisting of PLGA, PEG or PVA.

13. The method of claim 8, wherein administering includes administering nanoparticles wherein the at least one targeting peptide comprises AT2.

14. The method of claim 8, wherein administering includes administering nanoparticles wherein the at least one targeting peptide comprises at least one member selected from the group consisting of AT2, VCam1 or EpCAM.

15. The method of claim 8, wherein administering includes administering nanoparticles wherein the nanoparticle defines a hollow shell formed by the polymer, the at least one BCL-2/BCL-xl inhibitor is located within an interior of the hollow shell, and the at least one targeting peptide is coupled to an exterior surface of the hollow shell.

16. A composition of matter for targeted treatment of kidney fibrosis, comprising:
a plurality of nanoparticle comprising
at least one BCL-2/BCL-xl inhibitor;
at least one polymer comprising a member selected from the group consisting of PLGA, PEG or PVA; and
at least one targeting peptide,
wherein each of the plurality of nanoparticles defines a hollow shell formed by the polymer, the at least one BCL-2/BCL-xl inhibitor is located within an interior of the hollow shell, and the at least one targeting peptide is coupled to an exterior surface of the hollow shell.

17. The composition of claim 16, wherein the at least one BCL-2/BCL-xl inhibitor comprises at least one BCL-2 inhibitor selected from the group consisting of Navitoclax, ABT-737, Venetoclax or BM-1074.

18. The composition of claim 16, wherein the at least one BCL-2/BCL-xl inhibitor comprises at least one BCL-2 inhibitor selected from the group consisting of Navitoclax, ABT-737, Obatoclax Mesylate, TW-37, Venetoclax, BM-1074 or Gambogic Acid.

19. The composition of claim 16, wherein the at least one targeting peptide comprises AT2.

20. The composition of claim 16, wherein the at least one targeting peptide comprises at least one member selected from the group consisting of AT2, VCam1 or EpCAM.
